(19) United States
(12) Patent Application Publication
(10) Pub. No.: US 2022/0343784 A1
(43) Pub. Date: Oct. 27, 2022

(54) Methods and Systems for Sports and Cognitive Training

(71) Applicant: Brett Johnson, Beach Park, IL (US)

(72) Inventor: Brett Johnson, Beach Park, IL (US)

(21) Appl. No.: 17/641,200

(22) PCT Filed: Sep. 14, 2020
(86) PCT No.: PCT/US20/50730

§ 371 Date: Mar. 8, 2022

Related U.S. Application Data
(60) Provisional application No. 62/899,734, filed on Sep. 12, 2019.
(60) Provisional application No. 62/909,898, filed on Oct. 3, 2019.

(51) Int. Cl. G09B 5/06

(52) U.S. Cl. CPC G09B 5/065

(57) ABSTRACT

The disclosure provides example methods and non-transitory computer-readable mediums for providing randomized visual or audible stimuli for users to associate with corresponding physical movements. An example method includes a computing device (a) receiving from a user at least one selection from a plurality of stimuli in the form of audible or visual cues to determine parameters for a training program that includes a plurality of commands to the user to perform a corresponding activity, (b) providing the plurality of commands to the user, (c) responsively receiving from the user, via a feedback interface, one of a plurality of completion indications for the corresponding activity, and (d) concluding the training program based on a determination by the computing device that a threshold duration associated with the training program has been met or that a predetermined number of commands associated with the training program have been provided to the user.

### CROSS-REFERENCE TO RELATED APPLICATIONS

This application claims priority to U.S. Provisional Patent Application No. 62/899,734, filed on Sep. 12, 2019 and to U.S. Provisional Patent Application No. 62/909,898, filed on Oct. 3, 2019, which are hereby incorporated by reference in their entirety.

### BACKGROUND

Physical exercise devices that introduce some type of prompt that a user responds to are known in the art. The prompt in such devices is typically a light source, and the user must react to the light source, for example, through touch or movement into or out of the path of a light beam. Some systems have a number of these light sources that flash on and off in a predetermined sequence requiring the user to react accordingly or to physically move to different locations. The reaction time between activation of the light and the time it takes the person to react may also be measured as a performance metric.

### SUMMARY

In a first aspect, an example method for providing randomized visual or audible stimuli for users to associate with corresponding physical movements is disclosed. The method includes (a) receiving from a user, via a computing device, at least one selection from a plurality of stimuli in the form of audible or visual cues to determine parameters for a first training program, where the first training program includes a plurality of commands to the user to perform a corresponding activity; (b) providing, via the computing device, the plurality of commands to the user to perform the corresponding activity; (c) in response to each of the plurality of commands provided to the user to perform the corresponding activity, receiving from the user, via a first feedback interface communicatively coupled to the computing device, one of a plurality of completion indications for the corresponding activity performed by the user, where receiving from the user, via the first feedback interface communicatively coupled to the computing device, one of the plurality of completion indications for the corresponding activity performed by the user comprises the computing device detecting at least one of (i) a physical contact by the user on a touchscreen user interface of the computing device, (ii) a verbal cue from the user via a microphone of the computing device, (iii) a user's presence within a predetermined distance from the computing device via a proximity sensor, or (iv) a baseline image of a user via a camera of the computing device; and (d) concluding the first training program, via the computing device, based on a determination by the computing device that a threshold duration associated with the first training program has been met or that a predetermined number of commands associated with the first training program have been provided to the user.

In a second aspect, an example method for providing randomized visual or audible stimuli for users to associate with corresponding physical movements is provided. The method includes (a) receiving from a user, via a primary computing device, at least one selection from a plurality of stimuli in the form of audible or visual cues to determine parameters for a first training program, wherein the first training program includes a plurality of commands to the user to perform a corresponding activity; (b) syncing the first training program with a plurality of secondary computing devices via a wireless communication interface; (c) providing, via the primary computing device or one of the plurality of secondary computing devices, the plurality of commands to the user to perform the corresponding activity; (d) in response to each of the plurality of commands provided to the user to perform the corresponding activity, receiving from the user, via a first feedback interface communicatively coupled to at least one of the primary computing device or one of the plurality of secondary computing devices, one of a plurality of completion indications for the corresponding activity performed by the user, wherein receiving from the user, via a first feedback interface communicatively coupled to at least one of the primary computing device or one of the plurality of secondary computing devices, one of a plurality of completion indications for the corresponding activity performed by the user comprises at least one of the primary computing device or one of the plurality of secondary computing devices detecting at least one of (i) a physical contact by the user on a touchscreen user interface of the computing device, (ii) a verbal cue from the user via a microphone of the computing device, (iii) a user's presence within a predetermined distance from the computing device via a proximity sensor, or (iv) a baseline image of a user via a camera of the computing device; (e) concluding the first training program, via the primary computing device or one of the plurality of secondary computing devices, based on a determination by the primary computing device or one of the plurality of secondary computing devices that a threshold duration associated with the first training program has been met or that a predetermined number of commands associated with the first training program have been provided to the user.

In a third aspect, an example article of manufacture including a non-transitory computer-readable medium, having stored thereon program instructions that, upon execution by a computing device, cause the computing device to perform a set of acts according to the method of any one of the first aspect and the second aspect is disclosed.

In a fourth aspect, an example system is disclosed for providing randomized visual or audible stimuli for users to associate with corresponding physical movements. The system includes (a) a controller and (b) a non-transitory computer-readable medium, having stored thereon program instructions that, upon execution by the controller, cause the controller to perform a set of acts according to the method of any one of the first aspect and the second aspect is disclosed.

In a fifth aspect, an example non-transitory computer-readable medium having stored thereon program instructions that upon execution by a processor, cause performance of a set of acts according to the method of any one of the first aspect and the second aspect is disclosed.

### BRIEF DESCRIPTION OF THE FIGURES

FIG. 1A depicts an example graphical user interface (“GUI”) associated with a computing device, the GUI is configured to receive a user selection for a training program;

FIG. 1B depicts an example GUI configured to receive a user selection of one or more stimuli corresponding to commands for a training program;

FIG. 1C depicts the GUI of FIG. 1B reflecting the user selection of the stimuli;

FIG. 1D depicts a GUI configured to receive a user selection for a timed transition option between stimuli associated with the training program of FIGS. 1B-1C;

FIG. 1E depicts a GUI configured to receive a user selection of time duration options for the training program of FIGS. 1B-1D, specifically the “rounds” option is shown as selected and corresponds to the number of rounds of commands, including associated stimuli, that will be provided during a given training session;

FIG. 1F depicts a GUI configured to receive a user selection of time duration options for the training program of FIGS. 1B-1D, specifically the “countdown” option is shown as selected and shows the ability for the user to select the total duration of the training program;

FIG. 1G depicts a GUI summarizing the user selections for the training program of FIGS. 1B-1D and 1F;

FIG. 1H depicts a GUI displaying a countdown before the start of the training program based on the user selections received by the GUI corresponding to FIGS. 1B-1G;

FIG. 1I depicts a GUI with a countdown timer showing a command in the form of a color stimulus, the command corresponding to an activity for the user to perform;

FIG. 1J depicts a GUI with a countdown timer showing a command in the form of a number stimulus, the command corresponding to an activity for the user to perform;

FIG. 1K depicts a GUI according to FIG. 1J with a modified display of the number stimulus (e.g., in response to physical contact on a touchscreen) to reflect that the activity has been completed by the user and logged by the computing device;

FIG. 1L depicts an example GUI summarizing the user's performance metrics after concluding an example training program;

FIG. 2A depicts an example GUI configured to receive a user selection of one or more stimuli corresponding to commands for a training program;

FIG. 2B depicts a GUI configured to receive a user selection for a touch transition option between stimuli associated with the training program of FIG. 2A;

FIG. 2C depicts a GUI configured to receive a user indication of compliant data and non-compliant data;

FIG. 2D depicts a GUI with a countdown timer displaying a delay screen for transition between commands for the training program;

FIG. 2E depicts a GUI with a countdown timer showing a command in the form of a directional indicator, the command corresponding to an activity for the user to perform, the GUI configured to receive an indication of compliant data and non-compliant data for the training program;

FIG. 2F depicts a GUI displaying a second delay screen for transition between commands for the training program;

FIG. 2G depicts a GUI with a countdown timer showing a command in the form of a directional indicator, the command corresponding to an activity for the user to perform, as well as an indication that compliant data (e.g., “MADE”) was received by the computing device;

FIG. 2H depicts a GUI showing a command in the form of a directional indicator, the command corresponding to an activity for the user to perform, the GUI configured to receive an indication of compliant data and non-compliant data for the training program;

FIG. 2I depicts a GUI displaying an indication that compliant data (e.g., “SCORE”) was received by the computing device;

FIG. 2J depicts a GUI displaying a third delay screen for transition between commands for the training program;

FIG. 2K depicts a GUI showing a command in the form of a color stimulus, the command corresponding to an activity for the user to perform, the GUI configured to receive an indication of compliant data and non-compliant data for the training program;

FIG. 2L depicts an example GUI summarizing the user's performance metrics after concluding an example training program

FIG. 3A depicts a GUI configured to receive a user selection for a touch transition option between stimuli associated with an example training program, in particular a “touch anywhere” sub-option has been selected in this example;

FIG. 3B depicts a GUI showing a command in the form of a color stimulus, the command corresponding to an activity for the user to perform, the GUI configured to receive physical contact from a user to indicate completion of the activity;

FIG. 3C depicts a GUI displaying a delay screen for transition between commands for the training program;

FIG. 3D depicts a GUI showing a command in the form of a color stimulus, the command corresponding to an activity for the user to perform, the GUI configured to receive physical contact from a user to indicate completion of the activity;

FIG. 4 depicts a block diagram of a computing device and a computer network, according to an example implementation;

FIG. 5 shows a flowchart of a method, according to an example implementation; and

FIG. 6 shows a flowchart of a method, according to an example implementation.

The drawings are for the purpose of illustrating examples, but it is understood that the present disclosure is not limited to the arrangements and instrumentalities shown in the drawings.

### DETAILED DESCRIPTION

Examples of methods and systems are described herein. It should be understood that the words “exemplary,” “example,” and “illustrative,” are used herein to mean “serving as an example, instance, or illustration.” Any embodiment or feature described herein as “exemplary,” “example,” or “illustrative,” is not necessarily to be construed as preferred or advantageous over other embodiments or features. Further, the exemplary embodiments described herein are not meant to be limiting. It will be readily understood that certain aspects of the disclosed systems and methods can be arranged and combined in a wide variety of different configurations.

Example methods and non-transitory computer-readable medium having stored thereon program instructions that upon execution by a processor, cause performance of a set of acts, such as the method of the present disclosure (e.g., mobile app or application running in some other computing environment) are provided herein. For example, these methods and computer-readable mediums provide randomized visual and/or audible stimuli for users to associate with corresponding physical movements. The examples of the disclosure advantageously incorporate the brain and body in training sessions by causing users to perceive external information, to process that information to remember what movement the external information is associated with, and then to physically complete the movement as quickly and efficiently as possible.

Example Graphical User Interfaces

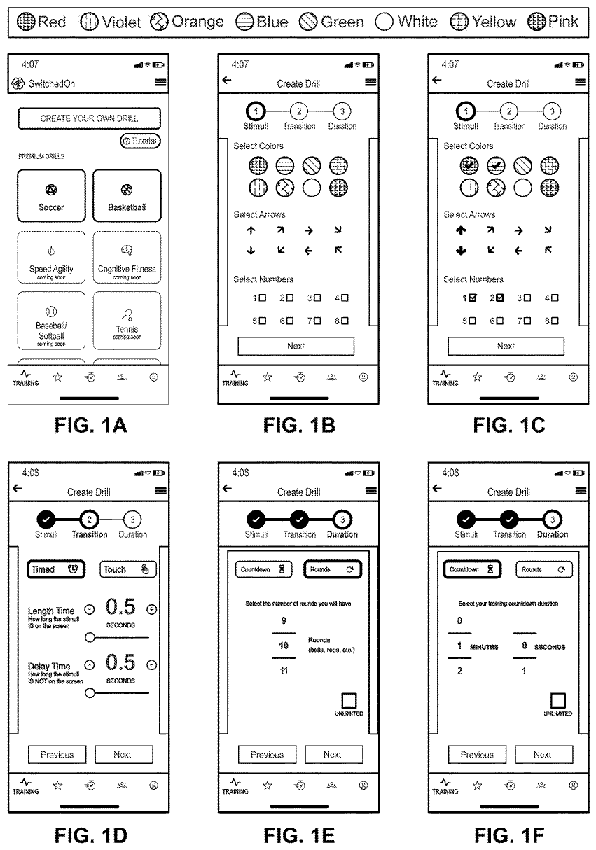

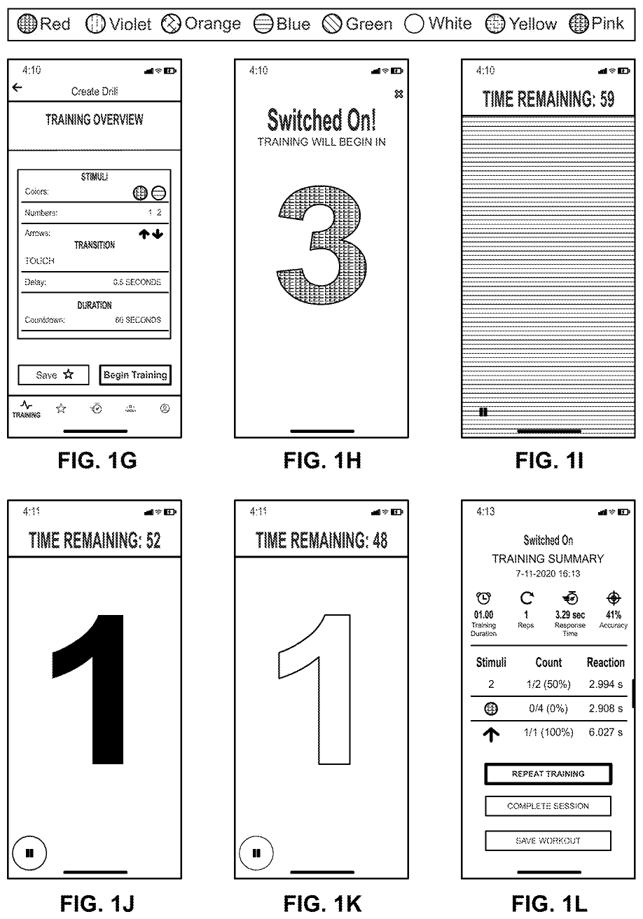

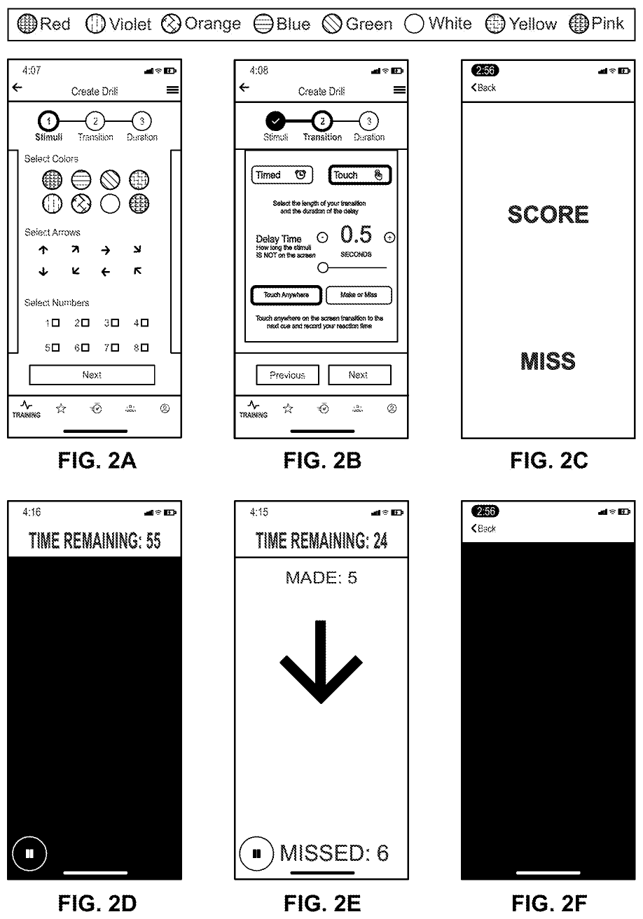

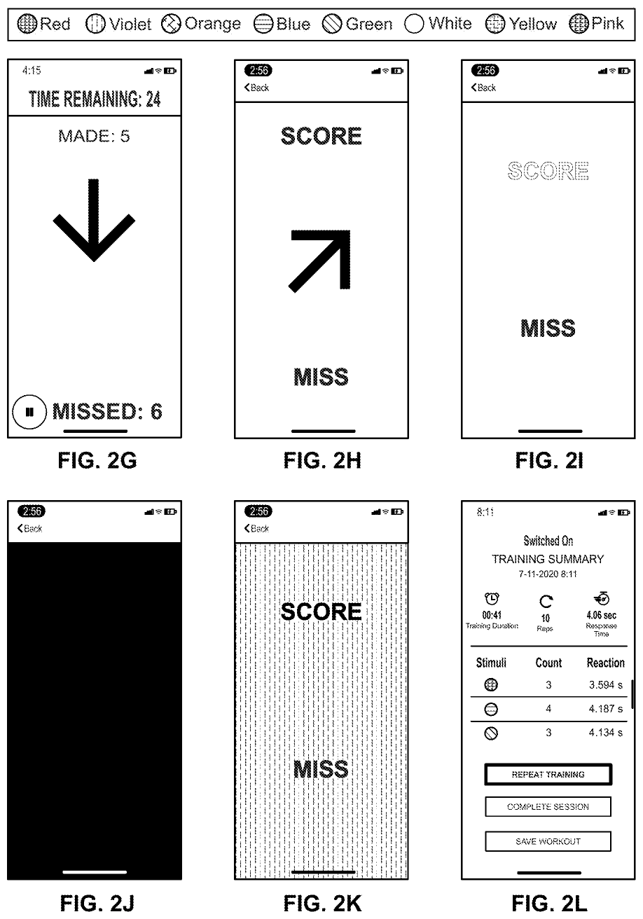

FIGS. 1A-1L and 2A-2L show example graphical user interfaces configured to be displayed on a computing device 200 to receive user selections, to provide commands to the user, and to receive completion indications for activities performed by the user, as some examples, in accordance with the methods and computer readable medium described below.

Example Architecture

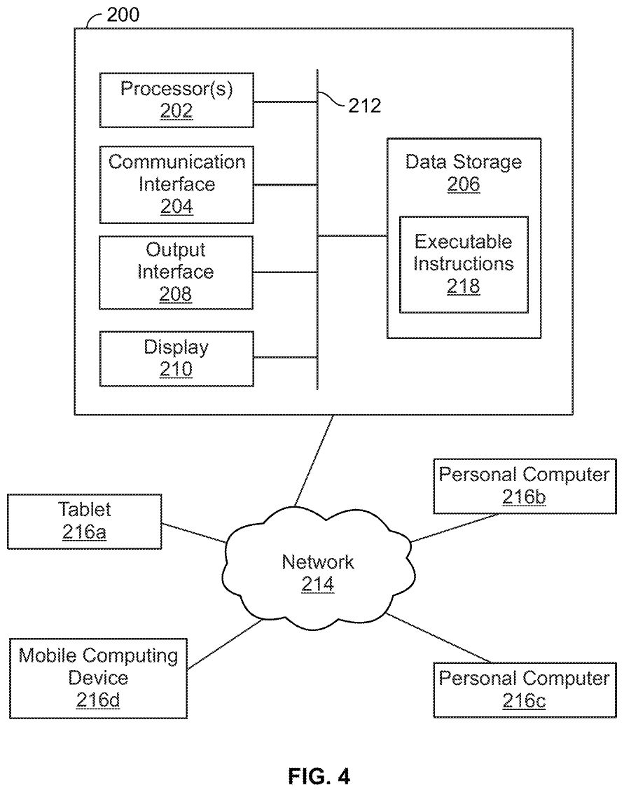

FIG. 4 is a block diagram illustrating an example of a computing device 200, according to an example implementation, that is configured to perform the methods described herein. For example, the computing device 200 can be a smartphone, tablet, or other mobile computing platform that includes a touchscreen user interface, a microphone, camera(s), IMUs, cellular data radios, WiFi radios, batteries, or other components.

The computing device 200 has a processor(s) 202, and also a communication interface 204, data storage 206, an output interface 208, and a display 210 each connected to a communication bus 212. The computing device 200 may also include hardware to enable communication within the computing device 200 and between the computing device 200 and other devices (e.g. not shown). The hardware may include transmitters, receivers, and antennas, for example.

The communication interface 204 may be a wireless interface and/or one or more wired interfaces that allow for both short-range communication and long-range communication to one or more networks 214 or to one or more remote computing devices 216 (e.g., a tablet 216a, a personal computer 216b, a laptop computer 216c and a mobile computing device 216d, for example). Such wireless interfaces may provide for communication under one or more wireless communication protocols, such as Bluetooth, WiFi (e.g., an institute of electrical and electronic engineers (IEEE) 802.11 protocol), Long-Term Evolution (LTE), cellular communications, near-field communication (NFC), and/or other wireless communication protocols. Such wired interfaces may include Ethernet interface, a Universal Serial Bus (USB) interface, or similar interface to communicate via a wire, a twisted pair of wires, a coaxial cable, an optical link, a fiber-optic link, or other physical connection to a wired network. Thus, the communication interface 204 may be configured to receive input data from one or more devices, and may also be configured to send output data to other devices.

The communication interface 204 may also include a user-input device, such as a keyboard, a keypad, a touch screen, a touch pad, a computer mouse, a track ball and/or other similar devices, for example.

The data storage 206 may include or take the form of one or more computer-readable storage media that can be read or accessed by the processor(s) 202. The computer-readable storage media can include volatile and/or non-volatile storage components, such as optical, magnetic, organic or other memory or disc storage, which can be integrated in whole or in part with the processor(s) 202. The data storage 206 is considered non-transitory computer readable media. In some examples, the data storage 206 can be implemented using a single physical device (e.g., one optical, magnetic, organic or other memory or disc storage unit), while in other examples, the data storage 206 can be implemented using two or more physical devices.

The data storage 206 thus is a non-transitory computer readable storage medium, and executable instructions 218 are stored thereon. The instructions 218 include computer executable code. When the instructions 218 are executed by the processor(s) 202, the processor(s) 202 are caused to perform functions. Such functions include, but are not limited to, the methods described elsewhere herein.

The processor(s) 202 may be a general-purpose processor or a special purpose processor (e.g., digital signal processors, application specific integrated circuits, etc.). The processor(s) 202 may receive inputs from the communication interface 204, and process the inputs to generate outputs that are stored in the data storage 206 and output to the display 210. The processor(s) 202 can be configured to execute the executable instructions 218 (e.g., computer-readable program instructions) that are stored in the data storage 206 and are executable to provide the functionality of the computing device 200 described herein.

The output interface 208 outputs information to the display 210 or to other components as well. Thus, the output interface 208 may be similar to the communication interface 204 and can be a wireless interface (e.g., transmitter) or a wired interface as well. The output interface 208 may send commands to one or more controllable devices, for example

Devices or systems may be used or configured to perform logical functions. In some instances, components of the devices and/or systems may be configured to perform the functions such that the components are configured and structured with hardware and/or software to enable such performance. Components of the devices and/or systems may be arranged to be adapted to, capable of, or suited for performing the functions, such as when operated in a specific manner.

It should be understood that methods disclosed herein are examples of functionality and operation of one possible implementation of the present examples. In this regard, each step may represent a module, a segment, or a portion of program code, which includes one or more instructions executable by a processor for implementing specific logical functions or steps in the process. The program code may be stored on any type of computer readable medium or data storage, for example, such as a storage device including a disk or hard drive. Further, the program code can be encoded on a computer-readable storage media in a machine-readable format, or on other non-transitory media or articles of manufacture. The computer readable medium may include non-transitory computer readable medium or memory, for example, such as computer-readable media that stores data for short periods of time such as register memory, processor cache and Random Access Memory (RAM). The computer readable medium may also include non-transitory media, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a tangible computer readable storage medium, for example.

Alternative implementations are included within the scope of the examples of the present disclosure in which functions may be executed out of order from that shown or discussed, including substantially concurrent or in reverse order, depending on the functionality involved, as would be understood by those reasonably skilled in the art.

The above description describes various features and functions of the disclosed systems, devices, and methods with reference to the accompanying figures. In the figures, similar symbols typically identify similar components, unless context indicates otherwise. The illustrative embodiments described in the detailed description, figures, and claims are not meant to be limiting. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

With respect to any or all of the message flow diagrams, scenarios, and flowcharts in the figures and as discussed herein, each step, block and/or communication may represent a processing of information and/or a transmission of information in accordance with example embodiments. Alternative embodiments are included within the scope of these example embodiments. In these alternative embodiments, for example, functions described as steps, blocks, transmissions, communications, requests, responses, and/or messages may be executed out of order from that shown or discussed, including in substantially concurrent or in reverse order, depending on the functionality involved. Further, more or fewer steps, blocks and/or functions may be used with any of the message flow diagrams, scenarios, and flow charts discussed herein, and these message flow diagrams, scenarios, and flow charts may be combined with one another, in part or in whole.

A step or block that represents a processing of information may correspond to circuitry that can be configured to perform the specific logical functions of a herein-described method or technique. Alternatively or additionally, a step or block that represents a processing of information may correspond to a module, a segment, or a portion of program code (including related data). The program code may include one or more instructions executable by a processor for implementing specific logical functions or actions in the method or technique. The program code and/or related data may be stored on any type of computer-readable medium, such as a storage device, including a disk drive, a hard drive, or other storage media.

The computer-readable medium may also include non-transitory computer-readable media such as computer-readable media that stores data for short periods of time like register memory, processor cache, and/or random access memory (RAM). The computer-readable media may also include non-transitory computer-readable media that stores program code and/or data for longer periods of time, such as secondary or persistent long term storage, like read only memory (ROM), optical or magnetic disks, and/or compact-disc read only memory (CD-ROM), for example. The computer-readable media may also be any other volatile or non-volatile storage systems. A computer-readable medium may be considered a computer-readable storage medium, for example, or a tangible storage device.

Moreover, a step or block that represents one or more information transmissions may correspond to information transmissions between software and/or hardware modules in the same physical device. However, other information transmissions may be between software modules and/or hardware modules in different physical devices.

Example Methods and Computer Readable Mediums

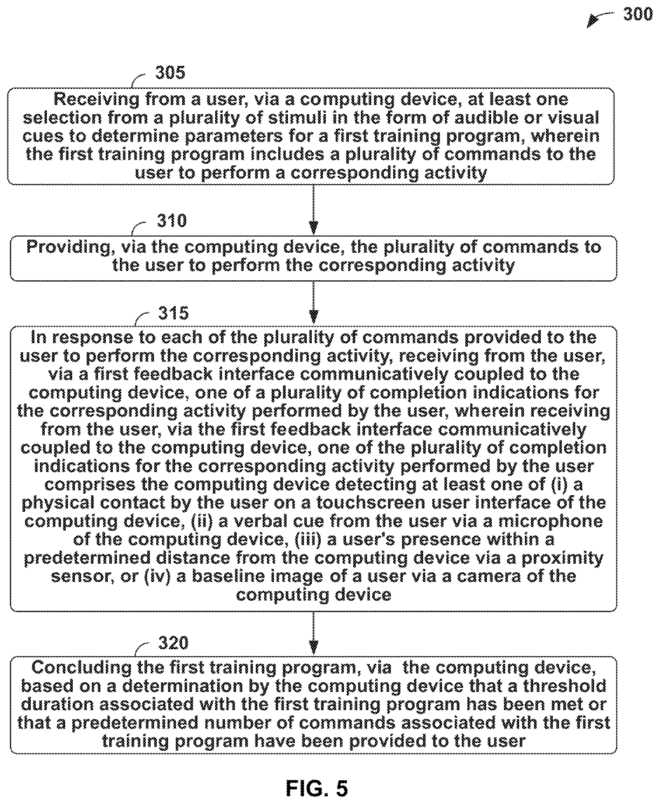

Referring now to FIG. 5, a method 300 for providing randomized visual or audible stimuli for users to associate with corresponding physical movements is illustrated using a computing device 200 of FIG. 4. Method 300 includes, at block 305, a computing device 200 receiving from a user, at least one selection from a plurality of stimuli in the form of audible or visual cues to determine parameters for a first training program, wherein the first training program includes a plurality of commands to the user to perform a corresponding activity. Then, at block 310, the computing device 200 provides the plurality of commands to the user to perform the corresponding activity. Next, at block 315, in response to each of the plurality of commands provided to the user to perform the corresponding activity, a first feedback interface communicatively coupled to the computing device receives from the user one of a plurality of completion indications for the corresponding activity performed by the user. And, at block 320, the computing device 200 concludes the first training program based on a determination by the computing device that a threshold duration associated with the first training program has been met or that a predetermined number of commands associated with the first training program have been provided to the user.

In this implementation, the step of receiving from the user, via the first feedback interface communicatively coupled to the computing device 200, one of the plurality of completion indications for the corresponding activity performed by the user includes the computing device 200 detecting at least one of (i) a physical contact by the user on a touchscreen user interface of the computing device, (ii) a verbal cue from the user via a microphone of the computing device, (iii) a user's presence within a predetermined distance from the computing device via a proximity sensor, or (iv) a baseline image of a user via a camera of the computing device. In one optional implementation, the proximity sensor could be an active motion sensor that puts out a signal in the form of ultrasonic waves, microwaves or a laser. Alternatively, the proximity sensor could be a passive motion sensor that receives infrared signals, for example, or the proximity sensor could be a camera.

In one optional example implementation, method 300 includes the computing device 200 determining a plurality of time differences between a time associated with providing each of the plurality of commands and a time associated with receiving each of the plurality of completion indications for the corresponding activity performed by the user. Then, based on the determined plurality of time differences, the computing device 200 determines, an average time difference. And the computing device 200 provides an indication of the determined average time difference to at least one of a display, a microphone, and a performance database. As used herein, the performance database may reside locally on the computing device 200 in data storage 206 or on one or more remote servers.

In various example implementations, users can select any combination of stimuli to appear during a training session. Users can further select whether to receive these stimuli in the form of audible and/or visual cues. In another implementation, the act of the computing device 200 providing the plurality of commands to the user to perform the corresponding activity includes at least one of (i) displaying the plurality of commands on a display or screen of the computing device, (ii) projecting the plurality of commands on a remote surface via the computing device, and (iii) issuing the plurality of commands as auditory cues via the computing device. In one example, the user may select one of the foregoing delivery methods of the stimuli. In another example, the screen of a computing device 200 may include, but is not limited to, the screen for a mobile phone, tablet, laptop, smart watch, etc.

In one implementation, the plurality of stimuli include one or more colors, numbers, directional indicators, words, or combinations thereof. Various examples of these stimuli are illustrated in the graphical user interfaces of FIGS. 1B-1C, 1I-1K, 2A, 2C, 2E, 2G-2I, 2K, 3B and 3D. In one optional implementation, the at least one selection from the user includes at least one color from a predetermined set of colors and at least one directional indicator (e.g., an arrow) from a predetermined set of directional indicators. In this example, the computing device 200 providing the plurality of commands to the user to perform the corresponding activity includes the computing device 200 displaying a first command as a color stimulus and the computing device 200 displaying a second command as a directional indicator.

In a further implementation, at least one of the plurality of commands includes an audible cue and a visual cue that are in conflict. In this optional example, the method includes the computing device 200 receiving from the user an indication that the user should perform the corresponding activity based on either the audible cue or the visual cue that are in conflict.

In another implementation, at least one of the plurality of commands includes a stimulus in the form of colored text. In this optional example, the computing device 200 receives from the user an indication that the user should perform the corresponding activity based on either a color of the colored text or a word command of the colored text. A computing device 200 may display “ColorText” stimulus such that a screen shows the color red, but the language of the text is the word “green.” An activity or user movement is associated with each of the colors red and green. A compliant user response would be to react to the color “red,” NOT the language “green” of the text. In one optional implementation, the computing device 200 may receive input from the user permitting customization of the text for the stimulus of various commands.

In another example of an audible cue and a visual cue in conflict, a computing device 200 may display the number “3,” but issue an audible command of “one” through a microphone, for example. And an activity or user movement is associated with both the number 3 and the number 1. Users must react to what they see visually and not to what they hear audibly. Conflicting audible and visual cues improve athlete training to perform better under pressure in sports. This is because pressure or stress can cause “choking” in sports by taking away focus from relevant information and placing it on irrelevant information. This type of training forces users to focus on a relevant piece of information, while simultaneously ignoring the irrelevant information, to improve similar cognitive processes. As a result, the methods of the present disclosure improve neurological responses in addition to improving physical performance and reaction time.

In one implementation, the first feedback interface is a microphone. In this example, the method 300 includes the microphone receiving a first indication that the user is in a baseline position based on a first verbal cue from the user. Then, responsive to receiving the first indication that the user is in the baseline position, the computing device 200 displays a first command to the user to perform a first activity. Next, the microphone receives a second indication that the user is in the baseline position based on a second verbal cue from the user. And, in response to the microphone receiving the second indication that the user is in the baseline position, the computing device 200 determines that the first activity has been completed.

In another example implementation, the computing device 200 receives from the user a first indication as to whether to display the plurality of commands for a predetermined length of time or a random length of time and a second indication as to whether to provide a delay between the display of the plurality of commands and a corresponding length of the delay between the display of each of the plurality of commands. In other words, the type of transition between the stimuli of the commands can be controlled by user selection. As shown in FIG. 1D, if the user selects a timed transition, the stimuli will transition automatically based on the amount of time chosen for the “length” and the “delay.” “Length” refers to the length of time that the stimulus will be displayed on the computing device 200, and “delay” refers to how long the stimulus will NOT be displayed on the computing device (e.g., a dark or blank screen will be shown). In the case for audible stimulus, the length will pertain to the amount of time it takes to say the given word. In one optional implementation, a user may select random length and delay times that will be presented as such: “_ to _ seconds.” This allows for a randomized display of the stimulus, as the stimulus will only appear during the chosen timeframe.

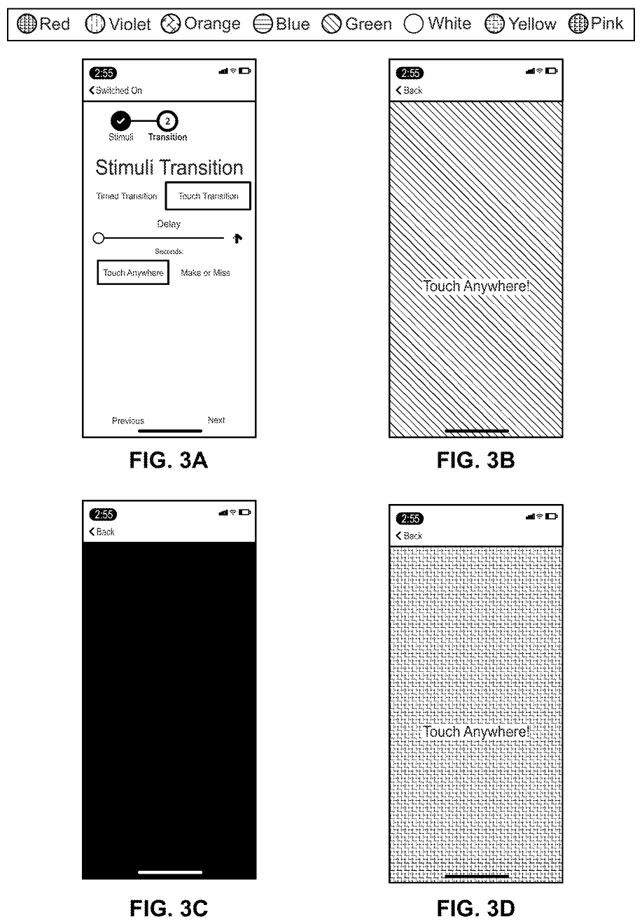

If the user chooses touch transition via a touchscreen of the computing device 200, the user initiates the stimulus transition to the delay screen by touching the screen. This touch feature can be used in the form of “touch anywhere” or “make or miss.” In order to initiate the transition, “touch anywhere” allows the user to touch anywhere on the screen of the computing device 200 (that is not already occupied by a button or space configured to receive alternative feedback), as shown in FIGS. 3A-D. This mode can also record data referred to as “reaction time,” as described in more detail below. As used herein, “reaction time” is the amount of time measured from the initial presentation of the stimulus until the touching of a screen by the user (or some other feedback mechanism described herein). Data for the entire training session will be received by the computing device 200 as feedback for historical reference, machine learning and AI-related applications, and the data can be correlated to each type of stimulus. In another embodiment, this data can be viewed and stored within a performance database that is either local to the computing device or on a remote server.

In an alternative transition option shown in FIGS. 2C-K, “make or miss” (i.e., pass or fail) allows the user to touch either “make” or “miss” in order to record “accuracy” or “compliance” data for an individual user's performance, as well as “reaction time” data. In addition, “make or miss” feedback will also initiate a transition between commands. This data may be provided for the entire training session and may be correlated to each command's stimulus. This data can be viewed and stored within a performance database local to the computing device 200 or on a remote server. In one example, the “make or miss” mode is designed to record the accuracy of a user's performance. For example, if a user selected a shooting drill in basketball (see FIG. 1A), after the user reacted to the stimulus and took the shot, the user would select either “Score” or “Miss” on the display of the computing device 200 to record whether the user made the shot or missed it. The corresponding reaction time would also be determined and stored in a performance database.

In another optional implementation, as shown in FIGS. 1I-K, 2D-E and 2G, a screen of the computing device displays a countdown meter that shows a visual representation of a time remaining before the computing device 200 provides a next command of the plurality of commands to the user to perform the corresponding activity. Alternatively, a user may select an option to use an “anticipation meter.” As used herein, the “anticipation meter” is a countdown meter that is displayed on the “delay” screen that gives a visual representation about how much time is left in the delay. The anticipation meter improves users' ability to anticipate when the stimulus will appear, which is an important skill to develop for activities like sports.

Voice recognition is another capability provided by the present disclosure. For example, users can activate transition of the command's stimulus by verbally identifying the stimulus (e.g., stating “RED”), and the computing device 200 will recognize the auditory feedback and then initiate transition of the stimulus to another command. Then a delay period will be initiated by the computing device 200, where the delay period can be customized by the user. The user can also select a “make” or “miss” option as a parameter of the first training program to permit auditory feedback to initiate transition similar to the make or miss touch feature used with the touchscreen.

In another optional implementation, a proximity sensor may be utilized by the computing device to receive feedback. For example, users can wave their hand a predetermined distance from the camera to initiate the transition. Then, the computing device 200 will initiate a delay period that can be customized by the user, as described above.

In one optional implementation, the first feedback interface communicatively coupled to the computing device 200 includes a proximity sensor. In this example, the method 300 may include the computing device 200 receiving an indication that a first position of the user is located a predetermined distance from the computing device 200. After the computing device 200 provides a first command to the user to perform a first activity, the proximity sensor monitors a current position of the user. Then, the proximity sensor determines that a second position of the user is located at the predetermined distance from the computing device 200. In response to a determination that the second position of the user is located at the predetermined distance from the computing device 200, the computing device 200 determines that the first activity has been completed.

In yet another implementation, the first feedback interface communicatively coupled to the computing device 200 includes a touchscreen user interface. In this example, method 300 includes the computing device 200 receiving a first indication that the user is in a baseline position based on a first physical contact by the user on the touchscreen. Then, responsive to receiving the first indication that the user is in the baseline position, the computing device 200 displays a first command to the user to perform a first activity. The computing device 200 then receives a second indication that the user is in the baseline position based on a second physical contact by the user on the touchscreen. And in response to the computing device 200 receiving the second indication that the user is in the baseline position, the computing device 200 determines that the first activity has been completed.

In a further implementation utilizing a touchscreen, the method 300 includes the computing device 200 determining a plurality of reaction times between a time associated with providing each of the plurality of commands to the user to perform the corresponding activity and a time associated with receiving a plurality of corresponding physical contacts on the touchscreen from the user. Then, the computing device 200 stores the plurality of reaction times on a performance database.

In yet another implementation utilizing a touchscreen user interface, the method 300 includes the computing device 200 determining whether a reaction time for each of a plurality of physical contacts from the user in response to each of the plurality of commands to the user to perform the corresponding activity is received within a threshold amount of time. This threshold amount of time may be pre-selected by the user as a parameter of the first training program. The computing device 200 then associates each reaction time for the plurality of physical contacts from the user that are received within the threshold amount of time as compliant data in the performance database. And the computing device 200 also associates each reaction time for the plurality of physical contacts from the user that are not received within the threshold amount of time as non-compliant data in the performance database. The computing device 200 stores the reaction times, the compliant data, and the non-compliant data in the performance database.

In another optional implementation, the method 300 includes the computing device 200 determining a second training program based on at least one of (i) the reaction times for each of the plurality of physical contacts from the user in response to each of the plurality of commands to the user to perform the corresponding activity, (ii) the compliant data points in the performance database, and (iii) the non-compliant data points in the performance database.

In a further optional implementation, users may select an option to transition between commands via physical contact (i.e., touching) with a specific stimulus (e.g., the color blue) on a touchscreen, while the other stimuli are on an automatic timer (e.g., directional indicators in the form of arrows). This is useful for training sessions that involve consistent activities or movements (e.g., 2-yard quick shuffles) followed by a user activity or movement that does not follow that pattern (e.g., a 20-yard sprint). This transition option stops the stimuli from transitioning until the user returns (e.g., from the 20 yard sprint) to the computing device 200 (e.g., mobile phone or tablet), where the user can provide feedback through the touchscreen to then initiate the delay screen and continue the training session.

In yet another optional implementation, the user can select an activity duration. If the user chooses a “countdown” duration, the length of the training session will be determined by a specific amount of time that may be displayed as a countdown timer on the training screen, as shown in FIGS. 1F-G, 1I-K, 2D-E, and 2G. The user can also choose “unlimited time” that may be displayed like a stopwatch on the training screen, counting up instead of down, for example.

In still another optional implementation, the user can select the “rounds” duration, as shown in FIG. 1E. In this mode, the length of the training session will be determined by a specific number of rounds, which refers to the number of stimuli that would appear during that training session. The number of rounds will be displayed during the training session counting down from the selected number of rounds chosen. The user can also choose “unlimited rounds” that would count up after each stimulus is presented.

In another optional embodiment, the first feedback interface includes a camera. In this example, the method 300 includes the computing device 200 receiving an indication that the user is in a baseline position. Then, responsive to receiving the indication that the user is in the baseline position, the computing device 200 operates the camera to obtain a first image of the user in the baseline position. After the computing device 200 provides a first command of the plurality of commands to the user to perform a first activity according to the first training program, the camera obtains a plurality of images of the user. Next, the computing device 200 continuously compares the first image of the user in the baseline position to each of the plurality of images of the user until the computing device 200 identifies a second image of the user in the baseline position. In response to the identification of the second image of the user in the baseline position, the computing device 200 determines that the first activity has been completed. For example, the camera can be operated to determine the starting position of the user, such that an automatic transition of the stimulus is initiated by the computing device upon completion of the user's physical movement and the user's return to that starting position.

In a further implementation, the method 300 includes the camera recording a video of the user for a duration of the first training program. The computing device 200 then stores the video of the user on a performance database. In yet another implementation, method 300 includes the computing device 200 modifying the video of the user to include at least one of a soundtrack, audio, a filter, or a slow-motion effect. In another implementation, the method 300 includes the computing device 200 selecting a video of a training session from another user and determining a plurality of parameters for the first training program based on the training session from another user

In another optional implementation, the method 300 further includes the computing device 200 providing a series of questions related to the first training program and receiving pre-training user feedback. Then, the computing device 200 determines the first training program based on the received pre-training user feedback. The computing device 200 also receives post-training user feedback. And the computing device 200 stores the pre-training user feedback and the post-training user feedback on a performance database. For example, a user may respond to a series of self-evaluation questions on a sliding scale, for example, that will be stored in by the computing device or sent to a database of a remote server. The responsive data may be displayed in different visual forms such as graphs, charts, etc. to provide feedback about the perceived effort of the user's performance. An example inquiry includes “How focused were you during your training session” and feedback may be received based on a scale from one to five.

In one optional implementation, a user may answer a series of written self-evaluation questions that will be stored in the data storage 206 of the computing device 200 and will permit users to document different aspects of their performance, as well as provide self-awareness. An example of these inquiries may include “What did I do that was good” with a blank space for the computing device 200 to receive written feedback.

In another optional implementation, a user may choose from a variety of pre-set training sessions that have all of the parameters described in the “custom start” feature that is predetermined in various combinations. This option may permit the user to view details of a training session that fits the user's needs and to start the training session with a single selection on the graphical user interface, which permits the user to forego customizing all of the settings. The user may also have the ability to optionally modify the settings of the selected training session.

An abundance of other information can be provided for each pre-set training session including but not limited to: video demonstration, starting position, distances, drill structure, set-up, how to increase physical/cognitive difficulty, etc.

In one optional implementation, a user may post the videos taken from their training session on a social media platform in communication with the computing device 200. Numerous filters, tabs, and groups may be available within a given platform.

In another optional implementation, users can see other user's posted training sessions, and choose to perform the same training session as those of other users with the same settings by via a single selection. This provides the ability to compete with others.

In other implementations, multiple users can sync their mobile computing devices to train simultaneously using multiple devices at once. These settings can be set up in many different ways, including but not limited to: all computing devices display the same stimulus or training program at the same time, all computing devices display completely random stimuli, only a single computing device provides a stimulus at one time, etc.

In one optional implementation, a coaching panel may be utilized. For example, one user or multiple users may view data from each individual computing device 200 to see the performance of each individual athlete or combined data from all of the computing devices 200, 216a-d.

In another implementation, a user may provide information like their goal and purpose and then be reminded by push notifications of their goals and purpose as frequently as they desire.

In one optional implementation, users can sync the computing device 200 to heartrate monitors and other wearable technology to receive data from these monitoring devices.

In one optional implementation, users can choose to add or associate specific training sessions to a database corresponding to their “favorites” that allows for a more convenient form of accessing and tracking training sessions that are repeatedly used or that users want to try, for example.

In another optional implementation, data from each training session can be stored on the computing device in a local performance database in the computing device's data storage 206 or a performance database on a remote server.

In a further optional implementation, users can provide feedback to the computing device 200 based on responses provided to a pre-training questionnaire to evaluate their energy level and required workload to determine what training session they should select and perform.

In yet another optional implementation, users can choose to show or share their training sessions with a coach who can provide online feedback.

In still another implementation, artificial intelligence can suggest users perform certain workouts based on prior activity including and not limited to: users' performance from previous training sessions, the type of training sessions users engaged in, users' written goals, etc.

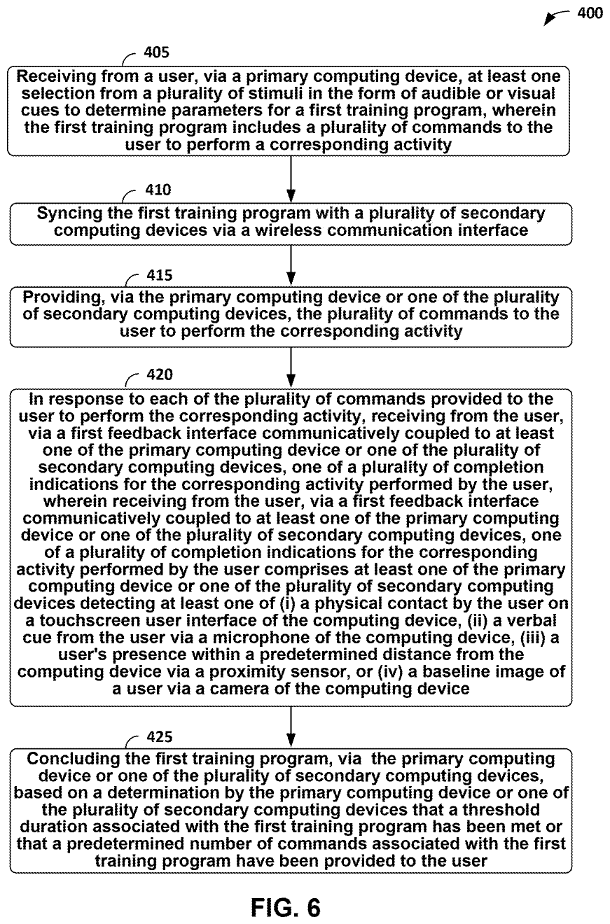

Referring now to FIG. 6, a method 400 for providing randomized visual or audible stimuli for users to associate with corresponding physical movements is illustrated using a computing device 200 and other devices 216a-d in the network 214 of FIG. 4. Method 400 includes, at block 405, a primary computing device 200 receiving from a user at least one selection from a plurality of stimuli in the form of audible or visual cues to determine parameters for a first training program. The first training program includes a plurality of commands to the user to perform a corresponding activity. Then, at block 410, the first training program is synced with a plurality of secondary computing devices 216a-d via a wireless communication interface 204 or network 214. Next, at block 415, the primary computing device 200 or one of the plurality of secondary computing devices 216a-d provides the plurality of commands to the user to perform the corresponding activity. Then, at block 420, in response to each of the plurality of commands provided to the user to perform the corresponding activity, a first feedback interface communicatively coupled to at least one of the primary computing device 200 or one of the plurality of secondary computing devices 216a-d receives from the user one of a plurality of completion indications for the corresponding activity performed by the user. And, at block 425, the primary computing device 200 or one of the plurality of secondary computing devices 216a-d concludes the first training program based on a determination by the primary computing device 200 or one of the plurality of secondary computing devices 216a-d that a threshold duration associated with the first training program has been met or that a predetermined number of commands associated with the first training program have been provided to the user.

In this implementation, the step of receiving from the user, via a first feedback interface communicatively coupled to at least one of the primary computing device 200 or one of the plurality of secondary computing devices 216a-d, one of a plurality of completion indications for the corresponding activity performed by the user includes at least one of the primary computing device 200 or one of the plurality of secondary computing devices 216a-d detecting at least one of (i) a physical contact by the user on a touchscreen user interface of the computing device, (ii) a verbal cue from the user via a microphone of at least one of the primary computing device 200 or one of the plurality of secondary computing devices 216a-d, (iii) a user's presence within a predetermined distance from at least one of the primary computing device 200 or one of the plurality of secondary computing devices 216a-d via a proximity sensor, or (iv) a baseline image of a user via a camera of at least one of the primary computing device 200 or one of the plurality of secondary computing devices 216a-d.

In one implementation, the present disclosure provides a non-transitory computer-readable medium having stored thereon program instructions that upon execution by a computing device 200, causes performance of a set of acts according to any of the foregoing methods.

In one implementation, the present disclosure provides an article of manufacture including the non-transitory computer-readable medium having stored thereon program instructions that upon execution by a computing device 200, causes performance of a set of acts according to any of the foregoing methods.

In one implementation, the present disclosure provides a system including a controller and the non-transitory computer-readable medium having stored thereon program instructions that upon execution by a computing device 200, causes performance of a set of acts according to any of the foregoing methods.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims.

We claim:

1. A method for providing randomized visual or audible stimuli for users to associate with corresponding physical movements, the method comprising:
receiving from a user, via a computing device, at least one selection from a plurality of stimuli in the form of audible or visual cues to determine parameters for a first training program, wherein the first training program includes a plurality of commands to the user to perform a corresponding activity;
providing, via the computing device, the plurality of commands to the user to perform the corresponding activity;
in response to each of the plurality of commands provided to the user to perform the corresponding activity, receiving from the user, via a first feedback interface communicatively coupled to the computing device, one of a plurality of completion indications for the corresponding activity performed by the user;
wherein receiving from the user, via the first feedback interface communicatively coupled to the computing device, one of the plurality of completion indications for the corresponding activity performed by the user comprises the computing device detecting at least one of (i) a physical contact by the user on a touchscreen user interface of the computing device, (ii) a verbal cue from the user via a microphone of the computing device, (iii) a user's presence within a predetermined distance from the computing device via a proximity sensor, or (iv) a baseline image of a user via a camera of the computing device; and
concluding the first training program, via the computing device, based on a determination by the computing device that a threshold duration associated with the first training program has been met or that a predetermined number of commands associated with the first training program have been provided to the user.

2. The method of claim 1, further comprising:
determining, via the computing device, a plurality of time differences between a time associated with providing each of the plurality of commands and a time associated with receiving each of the plurality of completion indications for the corresponding activity performed by the user;
based on the determined plurality of time differences, determining, via the computing device, an average time difference; and
providing, via the computing device, an indication of the determined average time difference to at least one of a display, a microphone, and a performance database.

3. The method of claim 1, wherein the at least one selection from the user comprises at least one color from a predetermined set of colors and at least one directional indicator from a predetermined set of directional indicators; and
wherein providing, via the computing device, the plurality of commands to the user to perform the corresponding activity comprises the computing device displaying a first command as a color stimulus and the computing device displaying a second command as a directional indicator.

4. The method of claim 1, further comprising:
receiving, via the computing device, an indication that the user is in a baseline position, wherein the first feedback interface comprises the camera;
responsive to receiving the indication that the user is in the baseline position, operating, via the computing device, the camera to obtain a first image of the user in the baseline position;
after providing, via the computing device, a first command of the plurality of commands to the user to perform a first activity according to the first training program, obtaining, via the camera, a plurality of images of the user;
continuously comparing, via the computing device, the first image of the user in the baseline position to each of the plurality of images of the user until the computing device identifies a second image of the user in the baseline position; and
in response to the identification of the second image of the user in the baseline position, determining, via the computing device, that the first activity has been completed.

5. The method of claim 4, further comprising:
recording, via the camera, a video of the user for a duration of the first training program; and
storing, via the computing device, the video of the user on a performance database.

6. The method of claim 5, further comprising:
modifying, via the computing device, the video of the user to include at least one of a soundtrack, audio, a filter, or a slow-motion effect.

7. The method of claim 1, further comprising:
receiving, via the computing device, an indication that a first position of the user is located a predetermined distance from the computing device, wherein the first feedback interface communicatively coupled to the computing device comprises the proximity sensor;
after providing, via the computing device, a first command to the user to perform a first activity, monitoring, via the proximity sensor, a current position of the user;
determining, via the proximity sensor, a second position of the user is located at the predetermined distance from the computing device; and
in response to a determination that the second position of the user is located at the predetermined distance from the computing device, determining, via the computing device, that the first activity has been completed.

8. The method of claim 1, wherein the first feedback interface communicatively coupled to the computing device comprises the touchscreen user interface, the method further comprising:
receiving, via the computing device, a first indication that the user is in a baseline position based on a first physical contact by the user on the touchscreen;
responsive to receiving the first indication that the user is in the baseline position, displaying, via the computing device, a first command to the user to perform a first activity;
receiving, via the computing device, a second indication that the user is in the baseline position based on a second physical contact by the user on the touchscreen; and
in response to receiving, via the computing device, the second indication that the user is in the baseline position, determining, via the computing device, that the first activity has been completed.

9. The method of claim 8, further comprising:
determining, via the computing device, a plurality of reaction times between a time associated with providing each of the plurality of commands to the user to perform the corresponding activity and a time associated with receiving a plurality of corresponding physical contacts on the touchscreen from the user; and
storing, via the computing device, the plurality of reaction times on a performance database.

10. The method of claim 1, wherein the first feedback interface communicatively coupled to the computing device comprises the touchscreen user interface, the method further comprising:
determining, via the computing device, whether a reaction time for each of a plurality of physical contacts from the user in response to each of the plurality of commands to the user to perform the corresponding activity is received within a threshold amount of time;
associating, via the computing device, each reaction time for the plurality of physical contacts from the user that are received within the threshold amount of time as compliant data in the performance database;
associating, via the computing device, each reaction time for the plurality of physical contacts from the user that are not received within the threshold amount of time as non-compliant data in the performance database; and
storing, via the computing device, the reaction times, the compliant data, and the non-compliant data in the performance database.

11. The method of claim 10, further comprising:
determining, via the computing device, a second training program based on at least one of (i) the reaction times for each of the plurality of physical contacts from the user in response to each of the plurality of commands to the user to perform the corresponding activity, (ii) the compliant data points in the performance database, and (iii) the non-compliant data points in the performance database.

12. The method of claim 1, wherein the first feedback interface comprises the microphone, the method further comprising:
receiving, via the microphone, a first indication that the user is in a baseline position based on a first verbal cue from the user;
responsive to receiving the first indication that the user is in the baseline position, displaying, via the computing device, a first command to the user to perform a first activity;
receiving, via the microphone, a second indication that the user is in the baseline position based on a second verbal cue from the user; and
in response to receiving, via the microphone, the second indication that the user is in the baseline position, determining, via the computing device, that the first activity has been completed.

13. The method of claim 1, wherein the plurality of stimuli comprise one or more colors, numbers, directional indicators, words, or combinations thereof.

14. The method of claim 1, wherein at least one of the plurality of commands comprises an audible cue and a visual cue that are in conflict, the method further comprising:
receiving from the user, via the computing device, an indication that the user should perform the corresponding activity based on either the audible cue or the visual cue that are in conflict.

15. The method of claim 1, wherein at least one of the plurality of commands comprises a stimulus in the form of colored text, the method further comprising:
receiving from the user, via the computing device, an indication that the user should perform the corresponding activity based on either a color of the colored text or a word command of the colored text.

16. The method of claim 1, wherein providing, via the computing device, the plurality of commands to the user to perform the corresponding activity comprises at least one of (i) displaying the plurality of commands on a screen of the computing device, (ii) projecting the plurality of commands on a remote surface via the computing device, and (iii) issuing the plurality of commands as auditory cues via the computing device.

17. The method of claim 1, further comprising:
receiving from the user, via the computing device, a first indication as to whether to display the plurality of commands for a predetermined length of time or a random length of time and a second indication as to whether to provide a delay between the display of the plurality of commands and a corresponding length of the delay between the display of each of the plurality of commands.

18. The method of claim 1, further comprising:
displaying, via a screen of the computing device, a countdown meter that shows a visual representation of a time remaining before the computing device provides a next command of the plurality of commands to the user to perform the corresponding activity.

19. The method of claim 1, further comprising:
providing, via the computing device, a series of questions related to the first training program;
receiving, via the computing device, pre-training user feedback;
determining, via the computing device, the first training program based on the received pre-training user feedback;
receiving, via the computing device, post-training user feedback; and
storing, via the computing device, the pre-training user feedback and the post-training user feedback on a performance database.

20. The method of claim 1, further comprising:
selecting, via the computing device, a video of a training session from another user; and
determining, via the computing device, a plurality of parameters for the first training program based on the training session from another user.
